SEQUENCE LISTING (13 sequences)

SEQ ID NO: 1 - DNA, 151 bp, artificial sequence
  n 19-32, 51-64, 83-96, 103-116, 147-151: n is g,a,t or c
tctagacagtctggagagnnnnnnnnnnnnnntctagacagtctggagagnnnnnnnnnnnnnntctagacagtctggagagnnnnnnnnnnnnnntctagannnnnnnnnnnnnnctctccagactgtctagacagtctggagagnnnnn

SEQ ID NO: 2 - DNA, 18 bp, artificial sequence
  Primer for biotinylation; biotinylated NeoTop2
ccgcttttctggattcat

SEQ ID NO: 3 - DNA, 21 bp, artificial sequence
  Adapter and primer for MAGE; UniXbaSD4 TopAdapter
tctagaggactgcgtgggcga

SEQ ID NO: 4 - DNA, 25 bp, artificial sequence
  n 24-25: n is g,a,t or c
cctcgcccacgcagtcctctagann

SEQ ID NO: 5 - DNA, 19 bp, artificial sequence
  Primer for MAGE, UniOligoPrimer4
cctcgcccacgcagtcctc

SEQ ID NO: 6 - DNA, 19 bp, artificial sequence
  Primer and adapter for SA-MAGE; SA-MAGE- TopAdapter
ttctctagacagtctggag

SEQ ID NO: 7 - DNA, 21 bp, artificial sequence
  n 20-21: n is g,a,t or c
ctccagactgtctagagaann

SEQ ID NO: 8 - DNA, 19 bp, artificial sequence
  Primer for SA-MAGE; SA-MAGE-BotPrimer
ctccagactgtctagagaa

SEQ ID NO: 9 - DNA, 18 bp, artificial sequence
  Primer for MAGE;
cggctgggtgtggcggac

SEQ ID NO: 10 - DNA, 16 bp, mus musculus
  exon from mouse albumin
tttctcagggtagcct

SEQ ID NO: 11 - DNA, 16 bp, mus musculus
  exon from HSP84
agctttgaattcatga

SEQ ID NO: 12 - DNA, 16 bp, mus musculus
  exon from actin binding protein
actacatctcctccct

SEQ ID NO: 13 - DNA, 16 bp, mus musculus
  exon from erythroid differentition regulatory protein
ggcgacacgcgcacct

(19) United States
(12) Patent Application Publication
(10) Pub. No.: US 2003/0143578 A1
(43) Pub. Date: Jul. 31, 2003

(54) High throughput method for identification of sequence tags

(76) Inventors: Steven C. Pruitt, Williamsville, NY (US); Lawrence M. Mielnicki, Buffalo, NY (US)

Correspondence Address: HODGSON RUSS LLP, SUITE 2000, ONE M&T PLAZA, BUFFALO, NY, 14203-2391, US

(21) Appl. No.: 10/227,719

(22) Filed: Aug. 26, 2002

Related U.S. Application Data
(60) Provisional application No. 60/314,991, filed on Aug. 24, 2001.

(51) Int. Cl. C12Q 1/68; G01N 33/53; C12P 19/34

(52) U.S. Cl. 435/6; 435/91.2; 435/7.5

(57) ABSTRACT

The present invention provides a method for rapid identification of sites of insertion of DNA in to a cellular chromosome. The present invention also provides a gene trap vector. In one embodiment, the method of the present invention comprises the steps of stably transfecting a population of cells with a gene-trap vector, identifying cells with a trapped gene, distributing sorted cells into a matrix format, pooling cells from the matrix into discrete pools, producing cDNA sequence tags from the trapped genes in the pooled cells, making concatamers for each pool, cloning and sequencing the concatamers, defining the sequence tag of each well in the matrix.

This application claims priority of U.S. Provisional application serial No. 60/314,991 filed on Aug. 24, 2001, the disclosure of which is incorporated herein by reference.

### FIELD OF THE INVENTION

This invention relates generally to the field of gene expression and more particularly to a method for high-throughput sequence tag identification based on modifications of the serial analysis of gene expression technology.

### BACKGROUND OF THE INVENTION

Following the near complete sequencing of the human genome, attention has now focused on the analysis of the expression and function of genes. Characterization of the expression status is of important for answering many biological questions. Changes in gene expression in response to a stimulus, a developmental stage, a pathological state or a physiological state are important in determining the nature and mechanism of the change and in finding cures for pathological conditions. Patterns of gene expression are also expected to be useful in the diagnosis of pathological conditions and may provide a basis for the subclassification of functionally different subtypes of cancerous conditions.

The function of a gene can be inferred from the effect of disrupting its sequence. A variety of means of disrupting the sequence of a gene are possible. One method, termed insertional mutagenesis, involves insertion of an additional sequence of DNA into the gene of interest. Insertional mutagenesis can be accomplished through several means including the use of natural viral sequences, or highly engineered gene sequence which confer additional functions at the insertion site. The use of an engineered sequence to integrate into a gene sequence is referred to as gene trapping (Skarnes et al., 1992, Genes Dev., 6:903-18; Durick et al., 1999, Genome Res., 9:1019-1025; Pruitt et al. 1992, Development 116:573-583,). In addition to disrupting the trapped gene, the engineered sequence may include reporter elements that allow its expression to be monitored.

A key step in the use of insertion mutagenesis or gene trapping is the identification of the gene into which the insertion event has occurred. Standard techniques using RACE or inverse PCR are currently used but are inefficient and limit the rate at which insertion sites can be identified. A method for rapid analysis of gene-expression (known as SAGE) has been proposed by Kinzler et al. (U.S. Pat. Nos. 5,695,937 and 5,866,330). This method involves identification of a short nucleotide sequence tag at a defined position in the mRNA. Concatamers are then formed from the short sequence tags and the tags are used to identify the mRNAs and the corresponding genes. However, this technique reports on all of the genes expressed within a cell and does not identify a specific gene into which an insertional event has occurred.

Accordingly, there is a need in the field of gene expression to develop methodologies whereby integration sites resulting from insertional mutagenesis techniques including gene trapping can be identified.

### BRIEF DESCRIPTION OF THE DRAWINGS

FIG. 1A is a schematic representation of the elements in one embodiment of the vector of the present invention.

FIG. 1B is a schematic representation of the integration of the gene-trap vector into a gene.

FIG. 2 is a schematic representation of the modified serial analysis of gene expression (MAGE) method of the present invention using the gene-trap vector of FIG. 1A. mRNA from the trapped gene is used to synthesize biotinylated cDNAs. The cDNAs are isolated via binding to strepavidin (or avidin) coated substrates, concatamers are formed following ligation with universal primers, followed by amplification and sequencing.

FIG. 3 is a schematic representation of an alternative method of the present invention—self amplifying MAGE (SA-MAGE) using the gene-trap vector of FIG. 1A. In this embodiment, PCR carried out using self-primers.

FIG. 4A is an illustration of the use of a 2×2×2 matrix format for defining column, row and stack sequence information.

FIG. 4B is an illustration of the use of a 3×3×3 matrix format for defining row, column and stack sequence information.

FIG. 5 is a schematic representation of SA-EGFP pA-PGK cassette excision. Expression of FLP recombinase by mating heterozygous animals with mouse strains expressing FLP recombinase will result in excision of a portion of the integrated sequence as shown. This region includes the SA, EGFP gene and pA site. Removal of the SA-EGFP-pA cassette then allows the 5′ endogenous gene splice donor to splice around the remaining promoterless NeoR gene reestablishing expression of a functional protein from the trapped gene. Any mutant phenotype observed in homozygous animals will be rescued following SA-EGFP-pA cassette excision.

FIG. 6 is a schematic representation of FLP-mediated re-integration into the original gene trap insertion sites.

FIG. 7 is a representation of the fluorescence distribution pattern for identification of cells by FACS in which a gene has been trapped.

FIG. 8 is a representation of the PCR products resulting from MAGE on cDNAs from a pool of gene trap cell lines. No products are observed in the control reactions (i.e., in the absence of RT)

FIG. 9 is a representation of the release of the sequence tag containing fragment from the MAGE PCR product for reactions digested with Xba1 (+) or not digested with Xba1(−). The markers are indicated on the left.

FIG. 10 is a representation of the template used to demonstrate SA-MAGE ligated to (+) SA-MAGE adapter (SEQ ID NO:6,7) or not ligated. The markers are indicated on the left.

FIG. 11 is a representation of concatamer formation during PCR using SA-MAGE after 30, 40 and 50 cycles with template amounts as indicated for ligated (l) and unligated (u) reactions.

FIG. 12 is a representation of SA-MAGE applied to concatamerization of sequence tags from a pool of gene trap cell lines. The lanes show electrophoresis of PCR products from FIG. 11 demonstrating the presence of concatamers in ligated (l) but not (u) reactions. No concatamers are seen in the control lane where RT was not used.

### SUMMARY OF THE INVENTION

The present invention provides a gene trap vector and a method for using the vector for rapid analysis of the site of integration for a large number of integration events using a high throughput screening method.

The gene trap vector of the present invention comprises elements for identification of integration events. These elements are splice acceptor site, a type IIS restriction endonuclease cleavage site (or other similar sites) and either a polyadenylation site or a splice donor In one embodiment the gene-trap vector comprises sequences representing gene-trapping functions, high throughput sequence tag acquisition and target gene modification. The sequences representing gene trap functions include, from 5′ to 3′, a splice acceptor, a series of termination codons in all three reading frame to ensure that the endogenous transcript codon does not occlude the internal ribosome entry site, an internal ribosome entry site, a nucleotide sequence encoding a reporter (such as one capable of directly or indirectly producing fluorescence), a poly-adenylation signal to terminate transcription, a promoter sequence, a selectable marker and a splice donor. The high throughput sequence tag acquisition components include a restriction endonuclease cleavage site allowing inclusion of sequences 3′ to the splice donor (such as a type IIS) integrated into or near the splice acceptor and splice donor. Further, recombinogenic sequences are present 5′ to the splice acceptor and between the promoter sequence (such as Pgk promoter) and selectable marker which permit modification of the trapped gene following incorporation of the gene-trap vector.

The method of the present invention comprises obtaining cells stably transfected with the gene-trap vector of the present invention; either pooling cells directly or distributing and expanding individual cells in a matrix format and pooling cells from defined sets of wells from the matrix, or pooling sorted cells based on expression levels from the trapped gene as reported by the reporter protein (such as a fluorescent protein reporter sequence using FACS); preparing mRNA from the pooled cells; synthesizing the first cDNA strands, synthesizing the second cDNA strands; isolating the DNA duplexes; digesting the duplexes with endonucleases to obtain Assay Tags comprising sequence tags unique to each trapped gene and a portion of the gene-trap vector; forming concatamers by either MAGE or SA-MAGE techniques described herein; cloning and sequencing the concatamers from each pool; and if desired, identifying the location of each sequence tag within the matrix.

The present invention also provides Assay Tags comprising a sequence tag from a trapped gene and a sequence from the gene-trap vector.

The present invention also provides kits for identification of sequence tags as described herein. The kits comprise one or more vials containing the gene-trap vector, a type IIS restriction endonuclease, primers for cDNA strand synthesis, PCR amplification or in the case of SA-MAGE, self amplification, and associated protocols.

### DESCRIPTION OF THE INVENTION

Definitions:

The term “Polynucleotide” as used herein means a polymeric form of nucleotides of at least 10 bases in length, either ribonucleotides or deoxyribonucleotides or a modified form of either type of nucleotide. The term includes single or double stranded form of DNA.

The term “Reporter Protein” or “reporter” is used interchangeably with “marker protein” or “marker” and as used herein means a protein produced from the transcription of a sequence of DNA present in the gene trap vector and which is detectable by an assay that does not depend on the endogenous gene's coding sequence that drives expression from the reporter protein.

The term “fluorescent reporter protein” or fluorescence reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins are GFP and EGFP whose presence in cells can be detected by flow cytometry methods.

The term “Trapped Gene” as used herein means a polynucleotide sequence in the genome of a cell which encodes for a protein and into which a polynucleotide sequence encoding the reporter/marker protein has been introduced.

The term “Vector” as used herein means a replicon, such as plasmid, phage or cosmid, to which another DNA segment may be attached so as to bring about the replication of the attached segment. A “vector” may further be defined as a replicable nucleic acid construct, e.g., plasmid or viral nucleic acid.

The term “Gene-Trap Vector” as used herein means a vector (such as plasmid) containing sequences allowing identification of integration events into genes. The Gene-trap vector comprises a splice acceptor, a type IIS restriction endonuclease cleavage site and a splice donor or a polyadenylation site. The gene-trap vector may also contain sequences allowing expression of a reporter gene from an endogenous gene's promoter when integrated into the endogenous gene. The vector may additionally contain sequence elements permitting splicing, termination of translation of the endogenous gene, internal ribosome entry, termination for transcription, insulator sequence elements, initiation of transcription, growth of cells in selective media, sequence specific recombination, or other elements.

The term “primer” as used herein refers to an oligonucleotide, whether occurring naturally or produced synthetically, which is capable of acting as a point of initiation of synthesis when placed under conditions in which synthesis of primer extension product which is complementary to a nucleic acid strand is induced, i.e., in the presence of nucleotides and an agent for polymerization such as DNA polymerase and at a suitable temperature and pH. The primer is preferably single stranded for maximum efficiency in amplification. Preferably, the primer is an oligodeoxy ribonucleotide. The primer must be sufficiently long to prime the synthesis of extension products in the presence of the agent for polymerization. The exact lengths of the primers will depend on many factors, including temperature and source of primer. The primers herein are selected to be “substantially” complementary to the different strands of each specific sequence to be amplified. This means that the primers must be sufficiently complementary to hybridize with their respective strands. Therefore, the primer sequence need not reflect the exact sequence of the template.

The term “Sequence Tag” or “sequence tag or tags as used herein means a sequence denoting a portion of the trapped gene.

The term “Assay Tags” or assay tag or tags used herein means a sequence comprising a Sequence Tag unique to a trapped gene and a portion of the gene-trap vector.

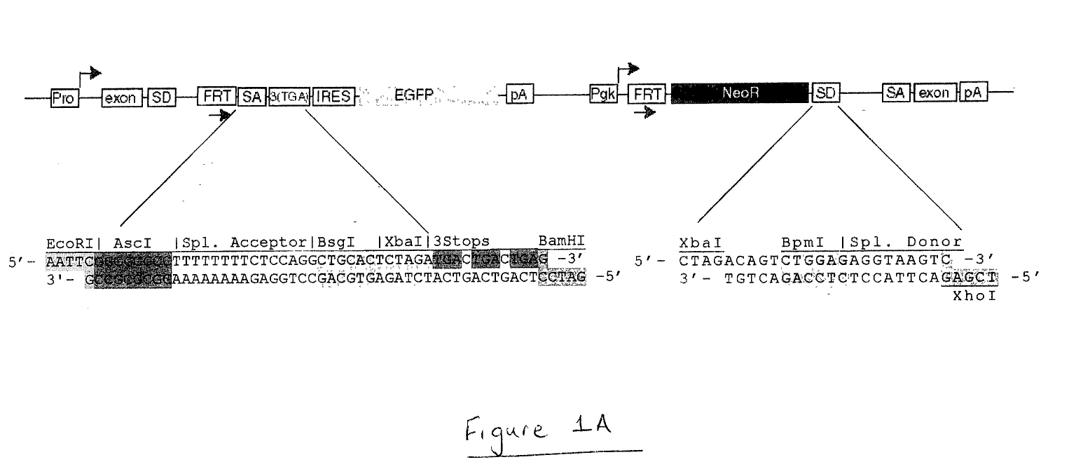

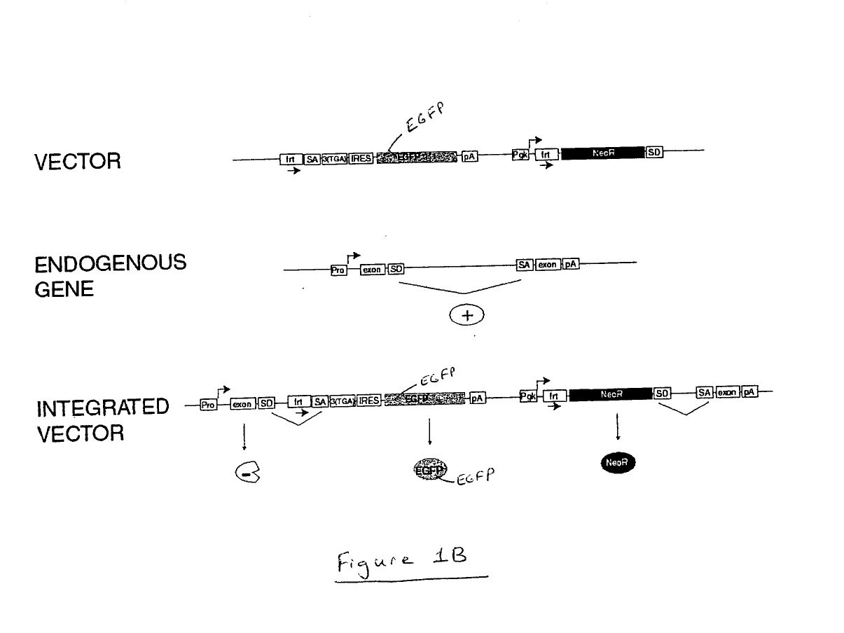

The present invention provides a gene-trap vector and a method for rapid analysis of gene expression using this vector. The method of the present invention is termed as modified serial analysis of gene expression or MAGE. One embodiment of the gene-trap vector has the overall structure shown in FIG. 1A. The vector includes elements allowing two discrete functions—1) gene-trapping functions, and 2) high throughput sequence tag acquisition. In one embodiment, the vector also includes one or more elements for allowing introduction of modifications to the structure of the integrated sequence subsequent to the initial gene-marking event.

1) Gene-Trapping Functions.

Elements for gene-trapping functions include a splice acceptor (SA) and a splice donor. Those skilled in the art will recognize that the splice donor can be replaced by a polyadenylation site. In one embodiment, a reporter coding sequence (such as the enhanced green fluorescent protein or EGFP) downstream of the splice acceptor is present such that, on integration into an intron of an endogenous gene, the reporter will become spliced into the endogenous message allowing its expression. In most cases, this also disrupts function of the endogenous gene. An internal ribosome entry site (IRES) is placed 5′ to the EGFP sequence to allow its expression regardless of the reading frame of the endogenous transcript. To insure that ribosomes initiating from the endogenous transcript start codon will not occlude the IRES or result in a fusion protein, a series of termination codons are placed 5′ to the IRES. The vector also carries a neomycin resistance gene driven from a constitutive promoter (Pgk) and followed by a splice donor to allow selection of stably transfected cell lines on integration into an endogenous gene.

2) High Throughput Sequence Tag Acquisition.

Elements allowing high efficiency acquisition of sequence tags are incorporated within the splice junctions. This is a key feature of the vector that permits a modified version of the Serial Amplification of Gene Expression (SAGE, Velculescu et al., 1995) technology to be utilized in identification of trapped genes in a high throughput format. This technology is referred to as MAGE or a variation SA-MAGE, and is described in detail below. The sequence elements allowing MAGE or SA-MAGE are the type IIS restriction endonuclease cleave sites incorporated at or near the splice acceptor and splice donor which in one embodiment described herein are BsgI and BpmI respectively. The type IIS enzymes recognize asymmetric base sequences and cleave DNA at a specified position up to about 20 base pairs outside of the recognition site. Other examples of type IIS restriction sites are BsmFI, MmeI and Fok1.

3) Target Gene Modification:

In one embodiment, to allow subsequent modification of sequences inserted into a trapped gene, FRT (FLP recombination target) sites are present in the vector. Although the use of FRT sites for this purpose is similar in principle to the use of the lox sites incorporated into the MSRHI (pGTlox2) gene trap vector, the positions of the FRT sites in the current vector allow greater flexibility in the modifications that can be subsequently incorporated. Placement of the recombinogenic sequences 5′ to the SA and 3′ to the promoter sequence (as illustrated in FIG. 1A) allows for the possibility of reconstitution of normal gene function from the trapped gene.

An example illustrating the elements of the gene-trap vector of the present invention are shown in FIG. 1. The vector comprises in downstream sequences: 1) A recombinogenic sequence element which in FIG. 1 mediates recombination by FLIP recombinase (frt) but which could comprise any sequence mediating recombination by a recombinase. Another example of such recombinogenic sequence elements are lox sites which mediate recombination by Cre recombinase. The preferred recombinogenic sites will contain half site mutations such that when two such half site mutations are recombined the double mutant site loses recombinogenic properties. 2) A splice acceptor sequence which in the present embodiment is based on a consensus splice acceptor. Alternative splice acceptor elements derived from natural or designed splice acceptors may be utilized. 3) A type IIS restriction endonuclease cleavage site, or any cleavage site allowing the inclusion of sequences 5′ to the splice acceptor. In the present embodiment a restriction endonuclease cleavage site for BsgI is utilized. The preferred sequence element will capture the maximum amount of 5′ adjacent sequence to facilitate gene identification. 4) Optionally, one or more translation termination sequences may be included where the preferred configuration of these sequence will be to terminate translation in alternative reading frames. The presence of translation termination sequences is preferred where an internal ribosome entry site is present 3′to these elements to prevent read through of the endogenous protein into the IRES 5) Optionally, an internal ribosome entry site may be included to facilitate ribosome re-entry and expression of a downstream gene. In the event that the translation termination sequences and/or IRES are omitted it is preferred to construct 3 alternative vectors such that the reading frame of the resulting read through product into a downstream gene will be systematically altered to include all possible coding frames. Embodiments of such vectors have been constructed here. 6) Optionally, a gene sequence may be included subsequent to the internal ribosome entry site. The preferred sequence will depend on the application but could include reporter proteins such as EGFP which is present in the current embodiment. Alternative reporter proteins could include other fluorescent proteins such as the red fluorescent protein (RFP) and the yellow fluorescent protein (YFP), proteins which are detectable via histochemical stains (e.g. β-galactosidase, alkaline phosphatase), proteins allowing positive selection (e.g. puromycin, blastocidin), proteins allowing negative selection (e.g. HSV-tk), proteins encoding recombinases (e.g. Cre, FLIP), proteins encoding transcription factors (e.g. TetON, TetOFF) or any other gene sequence that has a desirable function when expressed from the trapped gene promoter. Fusions between two proteins that confer the functions of each may also be used (e.g. β-GEO). 7) a polyadenylation signal may be included to terminate transcription from the endogenous gene promoter. This configuration is preferred where selection for insertion of the gene trap vector into non-expressed coding sequences is desired on the basis of a requirement for an endogenous 3′ polyadenylation signal. 8) Optionally, the vector may include an insulator sequence to prevent sequence elements downstream from influencing the endogenous genes promoter function. For example, the insulator may be the chicken β-globin insulator. 9) Optionally, a promoter element may be present which may be constitutively expressed as is the case for the Pgk promoter or may be inducible by specific agents or signals or tissue specifically expressed. 10) A recombinogenic sequence allowing recombination with the 5′ recombinogenic sequence. 11) a second gene sequence which may confer functions as described under 8 may be included. In the event that selection of non-expressed gene sequences is desired the preferred gene sequence will encode a selectable marker such as neomycin resistance which is included in the present embodiment. 12) A type IIS restriction endonuclease cleavage site or any cleavage site allowing the inclusion of sequences 3′ to the splice donor. In the present embodiment a restriction endonuclease cleavage site for BpmI is utilized. The preferred sequence element will capture the maximum amount of 3′ adjacent sequence to facilitate gene identification. An example of a sequence allowing capture of even more sequence than BpmI is MmeI. 13) A splice donor sequence. The sequences described above may be flanked by viral packaging sequences (e.g., retroviruses, adenoassociated virus) to facilitate introduction of the vector into cells.

The integration of the gene-trap vector into a gene is illustrated in FIG. 1B. Following introduction to the cell the vector sequence (Top) becomes integrated into an endogenous gene (Middle) leading to an integrated vector (Bottom). Following successful integration, the structure of the resulting sequence in the cell allows splicing of the vector sequence elements into the endogenous gene transcript. This results in expression from the endogenous gene promoter to create a bicistronic transcript encoding a portion of the original gene, translation of which is terminated within the vector. Ribosome re-entry occurs at the IRES to allow translation of EGFP. Transcription from the endogenous gene promoter is terminated by the polyadenylation signal. The Pgk promoter within the vector allows initiation of transcription regardless of the status of the endogenous gene. This transcript is spliced to the remainder of the endogenous gene via a splice donor. Transcripts from this promoter encode neomycin resistance.

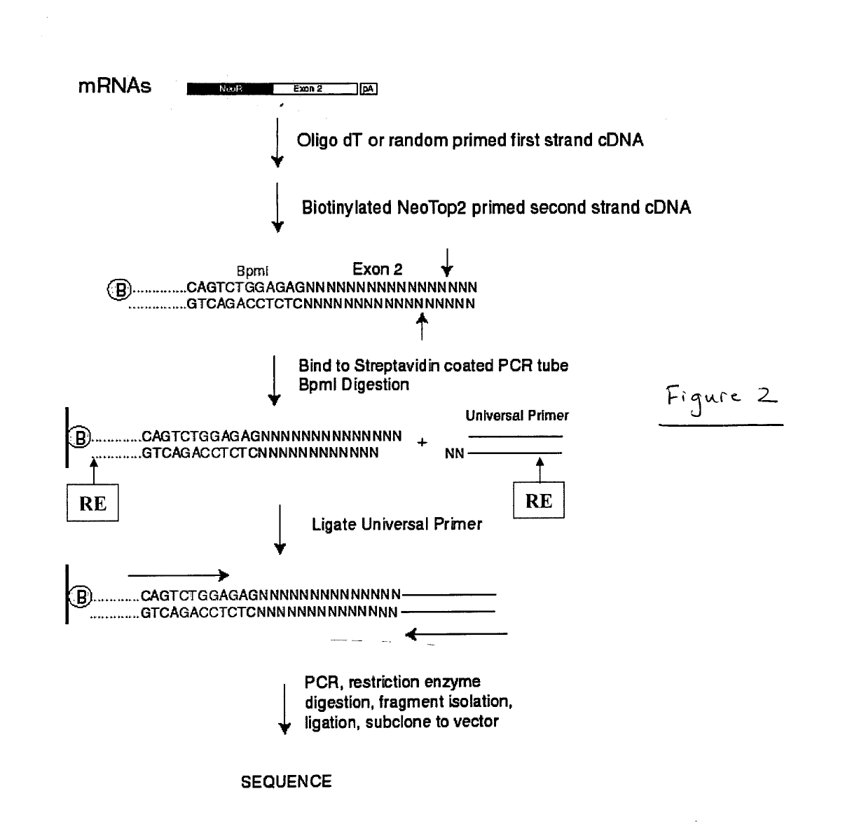

The vector of the present invention can be used in a modified SAGE (serial analysis of gene expression) method termed herein as MAGE. Modified SAGE technology (MAGE) is a high throughput method of identifying sequence tags resulting from gene trap vector integration events. The basis of this technology is shown in FIGS. 2-4. The first element on which it depends is the incorporation of recognition sites for restriction enzymes (REs) which cut distant to the recognition site itself. BsgI and BpmI are examples of such REs. FIGS. 1-4 show a vector with these recognition sites adjacent to the splice acceptor (SA) and splice donor (SD) elements within the gene trap vector. The restriction endonucleases BsgI and BpmI have the property wherein each cleaves the DNA at a position 16 nucleotides adjacent to the recognition sequence where the composition of the 16 nucleotides is irrelevant. As shown in FIG. 2, this property allows the amplification of either 15 or 14 nucleotides of the endogenous gene sequence adjacent to the SA and SD elements of the gene trap vector, respectively, which in turn allows differential amplification of endogenous gene sequence from cDNAs to messages that result from transcripts initiating from the endogenous gene promoter when BsgI is used or the Pgk promoter when BmpI is used. Hence, in the case of BsgI, the resulting products will reflect the relative expression level from the marked gene when assaying mixed pools, while BmpI will result in relatively even levels of amplification products.

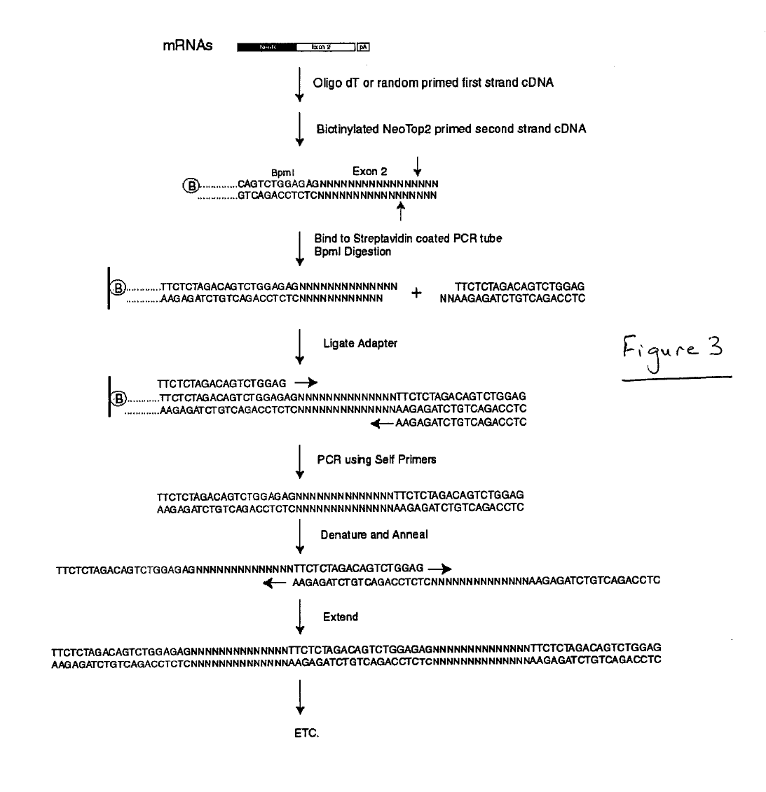

In the present invention, use is made of the application that it is possible to identify bits of unknown sequence information as long as these are separated by repeats of a known sequence. In the current application, this is accomplished by one of two alternative methods: MAGE in which the universal primer sequence is chosen to contain a restriction endonuclease cleavage site indicated as RE in FIG. 2, which in this illustration is Xba1, that is also present in the adjacent vector sequence allowing cleavage at this site, isolation of the resulting fragments containing the sequence tags and concatamerization mediated by ligation (FIG. 2) or self amplifying-MAGE (SA-MAGE) in which the universal primer is selected to generate a direct repeat of a portion of the vector sequence which then results in concatamerization mediated by self-priming events during the PCR step (FIG. 3). The ligated strings of sequence tags are then cloned and sequenced. In this way, sequences can be determined from each member present in a pool of marked genes. MAGE or SA-MAGE techniques can be used to identify sequence tags adjacent to either the splice acceptor or splice donor. Since transcripts expressed from the Pgk promoter will be present at relatively equal levels, use of SD junction fragments is desirable for determining all of the integration events within a pool of gene trap cell lines. Since transcripts from the endogenous gene promoter will reflect the expression level from that gene, use of the SA junction fragments is desirable for determining the relative levels of expression from different trapped genes. Data expected for MAGE from the splice donor site are shown below.

5′TCTAGACAGTCTGGAGAGNNNNNNNNNNNNNNTCTAGACAGTCTGGAG AGNNNNNNNNNNNNNNTCTAGACAGTCTGGAGNNNNNNNNNNNNNNTCTAGANNNNNNNNNNNNNNCTCTCCAGACTGTCTAGACAGTCTGGAGAG NNNN3′-(SEQ ID NO: 1)

Each repeating unit is 32 nucleotides long and contains 16 nucleotides that are derived from the vector/universal primer (TCTAGACAGTCTGGAG) (nucleotides 1-16 of SEQ ID NO.1) and 16 nucleotides that are derived from a discrete gene trap event (the splice donor AG plus 14 as underlined) and can be used to identify the insertion site. Inversion of the repeats is possible; however, this event is easily recognized by inversion of the vector/universal primer sequence (e.g. TCTAGA) (nucleotides 1-6 of SEQ ID NO:1) separating the tags. Similar data is expected for MAGE or SA-MAGE from either the splice acceptor or splice donor site except that the exact vector/universal primer sequences present in the string will differ.

MAGE or SA-MAGE can be used to define all of the insertion events in a pool of cells. Alternatively, by combining the ability to identify the sequence junctions resulting from multiple different gene trap events with a three-dimensional matrix strategy, a significant enhancement in the rate at which unique gene trap targets can be identified is also achieved.

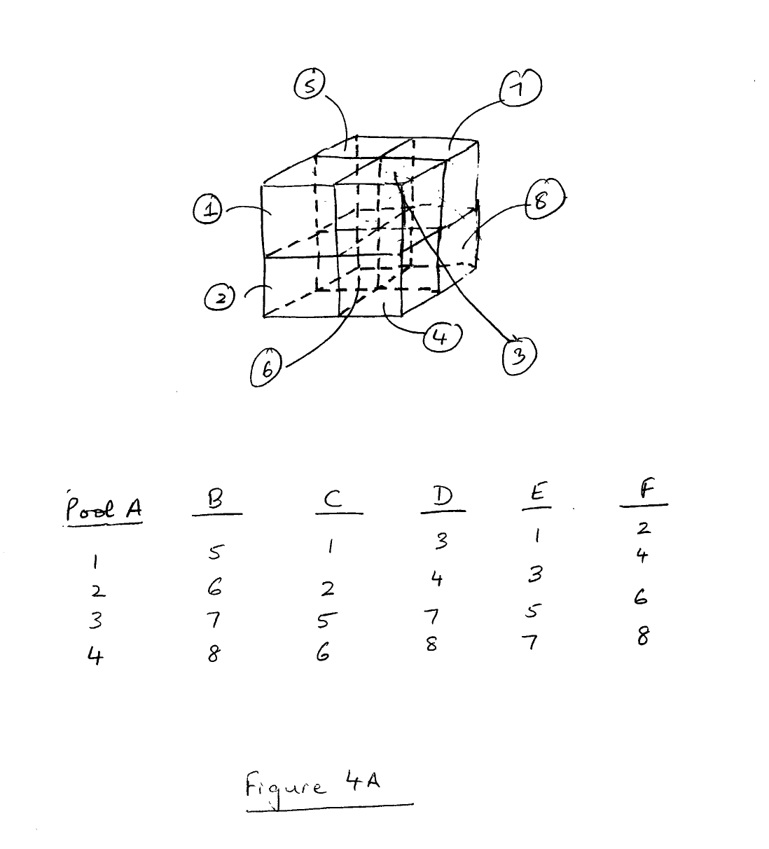

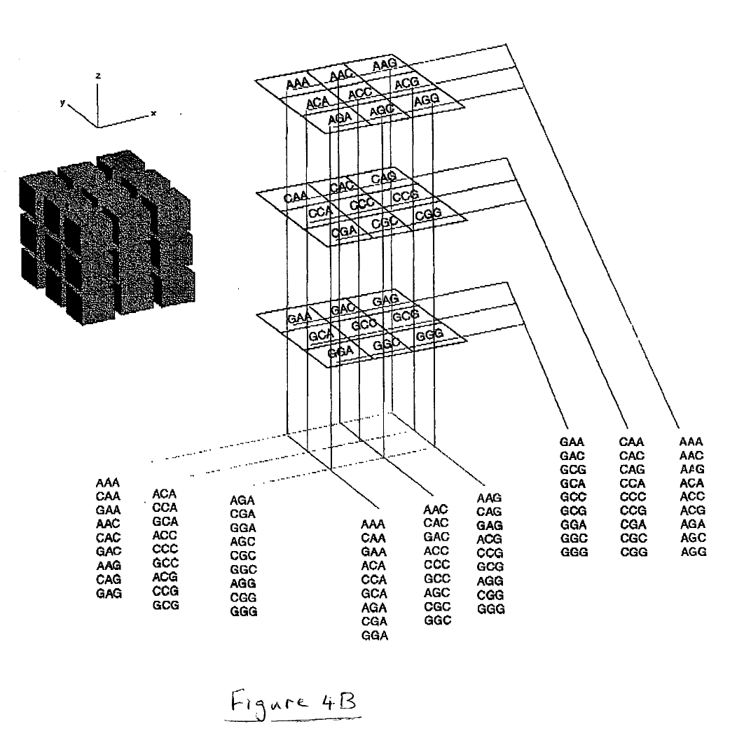

The matrix strategy involves the distribution of individual gene-trapped cells into discrete wells, which are present in a matrix format. An example of the usefulness of the 2×2×2 matrix format is shown in FIG. 4A. Assuming that each sequence is represented by a numeric identifier 1-8 corresponding to each well, the contents of the wells can be combined such that 6 pools A-F (4 wells per pool along the x, y and z planes) will define the location of all the contents of all the wells. Thus, if a sequence occurs in pools A, C and E, it can be traced back to well A and so on.

Another example of a matrix of the present invention utilizes a group of 27 different 3 nucleotide long sequences that are uniquely distributed to 27 different boxes in a 3×3×3 box format (FIG. 4B). By pooling samples from the boxes, 9 samples are derived that specify unique X, Y and Z coordinates within the matrix. To identify the location of a specific sequence within the matrix, the sequence is located within each X, Y and Z coordinate resulting in a unique row, column and stack position. In this small example, 9 pools of sequence information are sufficient to specify the location of 27 sequences.

To increase the efficiency, larger matrices may be used. For example, a 12×8×10 matrix can array 960 individual gene trap events in 10×96 well microtitre plates. Sequence information from a total of only 30 samples is then required to uniquely specify the marked sequence present in each of the 960 individual wells. Since a total of 32 nucleotides of sequence information is sufficient to define each target sequence, the length of sequence that will identify all of the information in a well containing 120 pooled samples is minimally 4,040 nucleotides. A 2.5 fold redundancy, or approximately 10,000 nucleotides of sequence per pooled sample, will insure that very few sequence tags are missed. Since approximately 500 nucleotides of sequence result from a single lane on an automated sequencing gel, generation of this amount of sequence can be accomplished in 20 lanes on a sequencing gel. The full 30 samples will require 600 lanes of information or the results from 6 sequencing gel cycles using a 100 lane format. Those skilled in the art will recognize that the x, y and z coordinates of the matrix of the present invention can independently have any value equal to or greater than 2 to see an effect on efficiency.

The method of the present invention for identification of insertion sites comprises the following steps: establishment of a pool of cells carrying the gene-trapped vector; isolation of RNA, synthesis of first cDNA strand; synthesis of a second (complementary) strand; digestion with a restriction endonuclease which cuts distant to the recognition site (a type IIS restriction endonuclease site) producing cDNA fragments (termed herein as Assay Tags) unique to each trapped gene; universal primer ligation; amplification of the Assay Tags by PCR; restriction endonuclease digestion, removal of competing DNA fragments and ligation of fragments to form concatamers in the case of MAGE (SA-MAGE does not require this step); cloning of the concatamers into an appropriate vector and transformation of host cells; DNA preparation and sequencing; definition of sequence tags; and deconvolution of matrix and assignment of specific sequence tag positions to individuals cells in the matrix.

For establishing pools of stably transfected cells, the gene-trap vector as described herein is used. The vector is randomly integrated into the genome of the target cell. Integration events into regions of the genome encoding functional genes are selected utilizing standard selection sites such as the neomycin resistance gene and based on the requirement for an endogenous poly-adenylation signal 3′ to the site of integration. Expression of the reporter protein is dependent on the endogenous gene promoter into which it is integrated and reflects the level of expression from this gene, providing a rapid vital cell marker by which expression from each trapped gene can be monitored. The design of the vector is such as to ensure that expression of the reporter protein will depend upon integration of the polynucleotide encoding it within protein-coding genes.

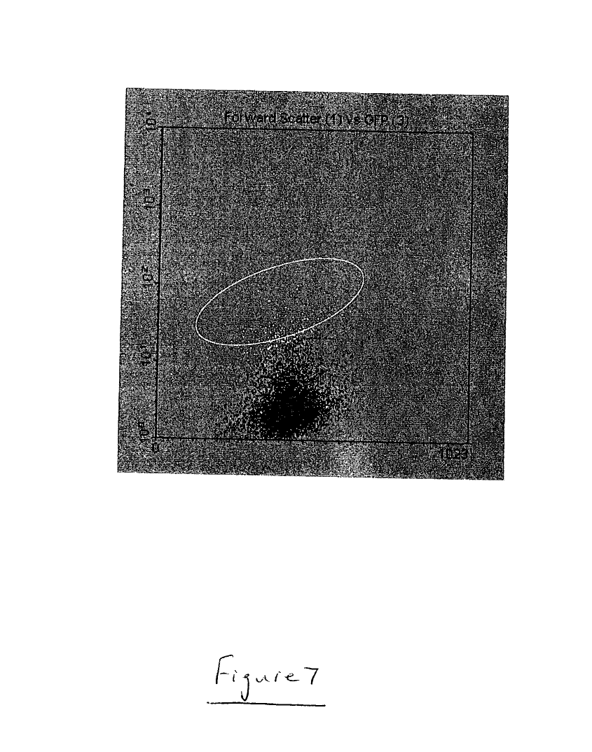

When cells are stably transfected with gene-trap vector comprising a reporter coding sequence, each cell carrying an endogenous gene marked by incorporation of the gene-trap vector is capable of reporting the expression from the endogenously marked gene. This property makes it possible to assess the expression of multiple genes simultaneously since populations of cells in which different endogenous genes are marked can be scored. One method by which this can be accomplished in an automated format appropriate for high-throughput analysis is by FACS. Thus, fluorescence activated cell sorting (FACS) is used to detect cells that express the fluorescent reporter and therefore identify the tagged gene. For a general overview of the FACS, see: Herzenberg et al., (Flow Cytometry, 1976, Sci. Amer. 234:108); Flow Cytometry and Sorting, (Eds., Malamad, Mullaney and Mendelsohn, John Wiley and Sons, Inc., New York, 1979). Briefly, fluorescence activated cell sorters take a suspension of cells and pass them single file into the light path of a laser placed near a detector. The laser usually has a set wavelength. The detector measures the fluorescent emission intensity of each cell as it passes through the instrument and generates a histogram plot of cell number versus fluorescent intensity (FIG. 7). Gates (windows) or limits can be placed on the histogram thus identifying a particular population of cells. FACS has the additional advantage of allowing the simultaneous isolation of responding cells.

The marked cell population can be sorted into the wells of a matrix type format to obtain colonies of cells in which a unique gene has been trapped. The cells from each discrete set of wells in the matrix can then be pooled to obtain well defined pools. For example, in a 3×3×3 matrix format, the number of pools are 9. The pooled cells are used for the preparation of mRNA.

Methods of extraction of RNA are well-known in the art and are described, for example, in J. Sambrook et al., “Molecular Cloning: A Laboratory Manual” (Cold Spring Harbor Laboratory Press, Cold Spring Harbor, N.Y., 1989), vol. 1, ch. 7, “Extraction, Purification, and Analysis of Messenger RNA from Eukaryotic Cells,” incorporated herein by this reference. Other isolation and extraction methods are also well-known. Typically, isolation is performed in the presence of chaotropic agents such as guanidinium chloride or guanidinium thiocyanate, although other detergents and extraction agents can alternatively be used.

Typically, the mRNA is isolated from the total extracted RNA by chromatography over oligo(dT)-cellulose or other chromatographic media that have the capacity to bind the polyadenylated 3′-portion of mRNA molecules. Alternatively, but less preferably, total RNA can be used. However, it is generally preferred to isolate poly(A)+RNA.

After preparation of the mRNA, the synthesis of the first cDNA strand is carried out. Methods of first strand complementary DNA synthesis are generally based upon the enzymatic synthesis of DNA from a nucleic acid template, e.g., messenger RNA. Enzymes capable of catalyzing the synthesis of DNA are referred to as “RNA dependent DNA polymerases” where the nucleic acid template is RNA and “DNA dependent DNA polymerases” where the template is DNA (generally, however, RNA dependent DNA polymerases are also capable of functioning as DNA dependent polymerases). More specifically, RNA dependent DNA polymerases such as AMV or MMLV reverse transcriptases are relied upon for the enzymatic synthesis of the first strand of complementary DNA from a messenger RNA template. Both types of DNA polymerases require, in addition to a template, a polynucleotide primer and deoxyribonucleotide triphosphates. The synthesis of first strand complementary DNA is usually primed with an oligo-d(T), consisting of 12-18 nucleotides in length, that initiates synthesis by annealing to the poly-A tract at the 3′ terminus of eukaryotic messenger RNA molecules. However, other primers, including short random oligonucleotide primers, can be used to prime complementary DNA synthesis. In the present embodiment the preferred method for priming first strand synthesis for use in MAGE or SA-MAGE from the splice acceptor is with a primer linked to an anchor molecule (such as biotin) containing sequences present in the region (within approximately 100 bp and with no intervening BsgI sites) 3′ to the splice acceptor from the reverse complement strand. This allows enrichment for sequences adjacent to the vector insertion site. During the polymerase reaction the primer is extended, stepwise, by the incorporation of deoxyribonucleotide triphosphates at the 3′ end of the primer. Additionally, for optimal activity, DNA polymerases usually require magnesium and other ions to be present in reaction buffers in well defined concentrations. The synthesis of the cDNA strand can be carried out by using commercially available kits (such as BRL Superscript II kit, BRL, Gaithersburg, Md.).

Following synthesis of the first strand of complementary DNA, several methods can be employed to replace the RNA template with the second strand of DNA. One such method involves removal of the messenger RNA with NaOH and self-priming by the first strand of complementary DNA for second strand synthesis. Generally, the 3′ end of single stranded complementary DNA is permitted to form a hairpin-like structure that primes synthesis of the second strand of complementary DNA by E. coli DNA polymerase I or reverse transcriptase. However, the method most commonly used involves the replacement synthesis of second strand complementary DNA. See, Gubler, U. and Hoffman, B. J. (1983) Gene 25: 263-269. During replacement synthesis the product of the first strand synthesis, a complementary DNA: messenger RNA hybrid, provides a template and a primer for a nick-translation reaction in which the enzyme RNase H produces nicks and gaps in the messenger RNA strand, resulting in a series of RNA primers for synthesis of the second strand of complementary DNA with the enzyme E. coli DNA polymerase I. In one embodiment the preferred method for priming second strand cDNA synthesis for use in MAGE or SA-MAGE from the splice donor is with a biotinylated primer containing sequences present in the region (within approximately 100 bp and with no intervening BmpI sites) 5′ to the splice donor from the reverse complement strand. This allows enrichment for sequences adjacent to the vector insertion site.

A preferred method for enriching for biotinlyated cDNAs following second strand cDNA synthesis is to incubate the cDNA on a streptavidin coated surface in a PCR tube or plate. Unbound cDNAs are removed by washing and the bound cDNAs are cleaved with either BsgI if cDNAs from the splice acceptor junctions are to be recovered or BpmI if cDNAs from the splice donor junctions are to be recovered. Un-biotinylated cleavage products are removed by washing.

Adapter ligation is accomplished by adding ligation buffer, ligase and the appropriate annealed universal adapter depending on whether the splice acceptor junction or splice donor junctions are to be amplified and whether MAGE or SA-MAGE is used.

For MAGE, the PCR amplified products are digested with XbaI in this embodiment, electrophoresed on a polyacrylamide gel and the sequence tag containing fragments are recovered and concatenated by ligation. SA-MAGE results in the formation of concatamers during the PCR amplification step and does not require the XbaI digestion, electrophoresis, recovery or ligation steps. After formation of concatamers, multiple sequence tags can be cloned into a vector for sequence analysis. Concatamers preferably contain sequence tags from about 15-20 genes. Analysis of the cloned concatamers is by standard sequencing methods.

Among the standard procedures for cloning the defined nucleotide sequence tags or concatamers of the invention is insertion of the tags into vectors such as plasmids or phage. The concatemers or Assay Tags produced by the method described herein are cloned into recombinant vectors for further analysis, e.g., sequence analysis, plaque/plasmid hybridization using the tags as probes, by methods known to those of skill in the art.

Vectors in which the Assay Tags or concatamers are cloned can be transferred into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

Transformation of a host cell with a vector containing the Assay Tags or the concatemers may be carried out by conventional techniques which are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed by electroporation or other commonly used methods in the art. The Assay Tags or concatamers present in a particular clone can be sequenced by standard methods (see for example, Current Protocols in Molecular Biology, supra, Unit 7) either manually or using automated methods.

Once the sequence of the concatamers from different sets of pools from the matrix is obtained, the location (i.e., the x, y and z coordinates) of a particular sequence tag within the matrix can be identified based on the occurrence of the sequence tags in different sets of pools. As discussed herein, the use of the matrix format reduces the number of samples which need to be cloned and sequenced to obtain information on the sequence of the entire population of trapped genes.

Once cells are identified in which the trapped gene of interest is present, standard molecular biology techniques can be used to confirm the identity of the trapped gene if desirable. Techniques such as inverse PCR or RACE can be used for this purpose and are well known to those skilled in the art. The PCR based techniques take advantage of the known portion of the fusion transcript sequence (Frohman et al., 1988, Proc. Natl. Acad. Sci., USA., 1988:8998-9002). Typically, such sequence is be encoded by the foreign exon containing the selectable marker/reporter. The first step in the process generates single stranded complementary DNA which is used in a PCR amplification reaction. The RNA substrate for cDNA synthesis may either be total cellular RNA or an mRNA fraction, preferably the latter. mRNA is isolated from cells lysed and mRNA is bound by the complementary binding of the polyadenylate tail to a solid matrix-bound polythymidine. The bound mRNA is washed several times and the reagents of the reverse transcription (RT) reaction are added. cDNA synthesis in the RT reaction is initiated at random positions along the message by the binding of a random sequence primer (RS). This RS primer has 6-9 random nucleotides at the 3′end to bind sites in the mRNA to prime cDNA synthesis, and a 5′ tail sequence of known composition to act an anchor for PCR amplification in the next step. There is therefore no specificity for the trapped message in the RT step. Alternatively, a poly-dT primer appended to the specific sequences for the PCR may be used. Synthesis of the first strand of the cDNA would then initiate at the end of each trapped gene. In the next step, PCR amplification is used. The primers for this reaction are complementary to the anchor sequence of the RS primer and to the selectable marker. Double stranded fragments between a fixed point in the selectable marker gene and various points downstream in the appended transcript sequence are amplified. These fragments subsequently become substrates for DNA sequencing reactions.

The ability to manipulate the sequence carried at the site of integration in a gene trap line is a useful feature. The present technology is an improvement over that of Hardouin and Nagy, 2000 (Genesis. April; 26(4):245-52.); and Araki et al., 1997 (Nucleic Acids Res. February 15; 25(4):868-72) in that it allow greater utility in subsequent modifications. Specifically, the placement of the recombinogenic sequences allows modifications to be made that will permit greater utility and unique applications.

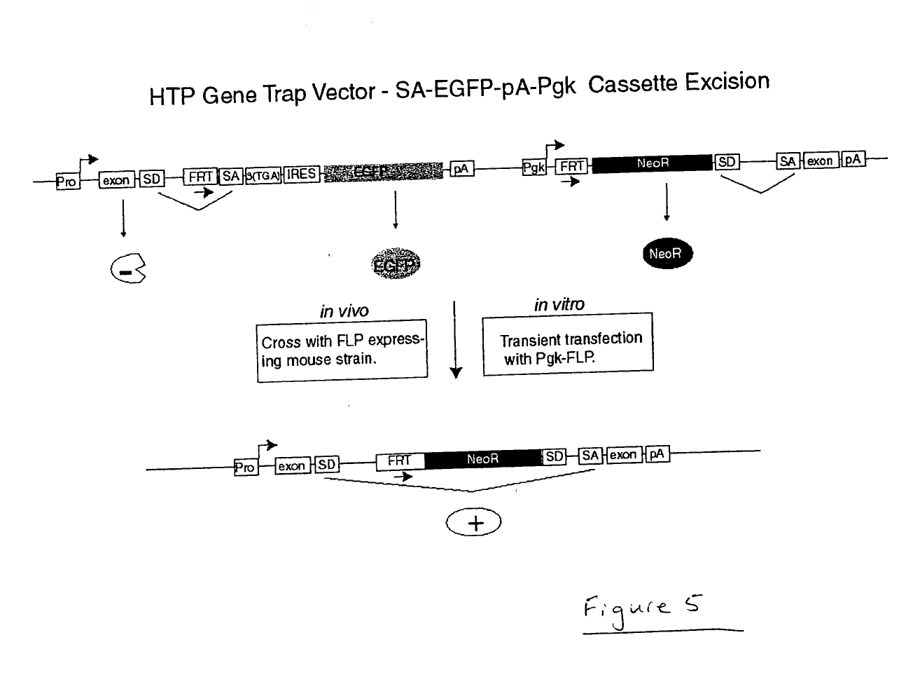

An example of a use of the gene trap vector is in determining the phenotypes associated with disruption of the endogenous genes into which the vector has become integrated in mice. Generally, the phenotype will be manifest in homozygous, but not heterozygous, animals and often it will be homozygous lethal. Expression of FLP recombinase by mating heterozygous animals with mouse strains will result in excision of a portion of the integrated sequence as shown in FIG. 5. This region includes the SA, EGFP gene and pA site. FLP has already been used successfully to mediate FRT dependent recombination in ES cells and mice (Dymecki, 1996, Proc Natl Acad Sci U S A. June 11; 93(12):6191-6; Dymecki and Tomasiewicz, 1998, Dev Biol. September 1; 201(1):57-65). Removal of the SA-EGFP-pA cassette then allows the 5′ endogenous gene splice donor to splice around the remaining promoterless NeoR gene reestablishing expression of a functional protein. The mutant phenotype in homozygous animals can be rescued following SA-EGFP-pA cassette excision.

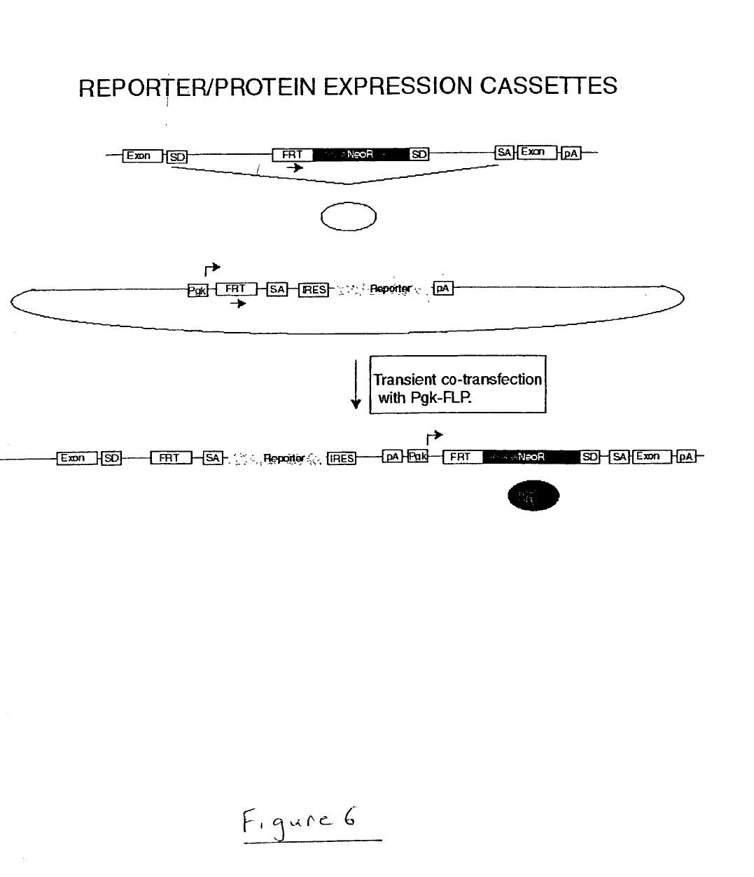

In another embodiment, as shown in FIG. 5, it is possible to remove the SA-EGFP-pA-Pgk cassette either in vivo or by transient transfection of cell lines with a Pgk-FLP expression vector in the original ES cell clone in vitro. Although there is no selection for loss of this cassette (unless EGFP is expressed from the endogenous gene promoter in ES cells), this can be accomplished effectively by transient co-transfection of the Pgk-FLP expression vector with an EGFP expression construct and FACS sorting (e.g. Gagneten et al., 1997, Nucleic Acids Res. August 15; 25(16):3326-31). Removal of the SA-EGFP-pA-Pgk cassette results in loss of neomycin resistance. This allows use of G418 selection in subsequent FLP mediated re-integration events as shown in FIG. 6. This methodology can be utilized to introduce a variety of additional gene sequences, bringing their expression under control of the endogenous gene promoter and enhancer elements. These may include alternative reporters, Cre-recombinase, and, perhaps more importantly, genes encoding proteins designed for specific applications within the context of a given experimental paradigm.

In another embodiment, the present invention provides a kit useful for detection of sequence tags. The kit comprises one or more vials or container comprising a gene-trap vector as provided herein, universal primers containing type IIS restriction endonuclease and protocols.

The present invention also provides Assay tags comprising a part of the gene-trap vector and a part of the trapped gene. The part of the Assay Tag which is the part of the gene-trap vector is a type IIS restriction endonuclease site The Assay Tags may reflect a function of interest that is mediated by the insertion event. An example of such a function would be the induction of tumorigenesis or altered physiological state.

The present invention also provides cell lines or libraries of cell lines which are marked by integration of the gene trap vector and which may be pools of cells or arrayed in matrices.

The present invention also provides a protocol of concatamerization and amplification of a sequence of DNA and any intervening sequence through the ligation of a direct repeat of that sequence and PCR regardless of whether the sequence is carried by a vector.

The present invention will be further understood by the examples presented below, which are to be construed as illustrative and are not intended to be restrictive in any way.

### EXAMPLE 1

This embodiment describes the construction of the gene-trap vector. In the current embodiment the vector comprises sequences assembled through a series of standard molecular biology techniques from commercially available DNA constructs, synthetic oligonucleotides, and constructs previously constructed by the inventor (are these disclosed somewhere so that they can be referenced?). Elements shown in FIG. 1A spanning the EcoRI site through the BamHI site of the sequence shown to the left and including the splice acceptor, BsgI site, XbaI site, translation termination signals and BamHI site as well as elements shown in the sequence to the right spanning the XbaI site through the XhoI site and including the BpmI site and splice donor sequences were synthesized as oligonucleotides. The IRES, EGFP and pA sequences shown in FIG. 1B (vector) were purchased (ClonTech). The Pgk promoter fragment is from the construct PgkvecR and was originally derived from the construct pTI (Skames et al., 1992).

Five different versions of the gene trap vector have been constructed. The first generation gene trap vector, pHTP-GT, contains the destabilized, red-shifted variant of GFP from Aequorea victoria, d2EGFP (ref). This vector was made using several pre-existing plasmids, pd2EGFP and pIRESNEO (Clontech, Palo Alto, Calif.), and the pGK promoter from pT1 (Skarnes et al. 1992), as well as sequences specifically synthesized for these constructions. To start, the d2EGFP encoding sequences of pd2EGFP were removed by BamHI (filled in) and XbaI digestion and used to replace the SmaI to XbaI Neomycin phosphotransferase encoding portion of pIRESNEO, resulting in pIRESd2EGFP. A synthetic double stranded splice acceptor (SA) containing DNA oligonucleotide with BamHI and SphI overhangs was used to replace the IVS sequence in pIRESd2EGFP between those same sites, resulting in the plasmid pSA-IRESd2EGFP. This construct was linearized with XhoI, blunted ended, ligated to a double stranded blunt-ended NotI DNA linker and subsequently digested with NotI, to isolate a 1.3 kb SA-ires-d2EGFP-pA containing NotI fragment.

A plasmid containing the pGK promoter from pT1 (Skarnes et al., 1992) and Neo from Clonetech was modified by insertion of a synthetic double stranded splice donor (SD) containing DNA oligonucleotide with XbaI and XhoI overhangs downstream of the PGKNeo cassette replacing the bovine growth hormone polyadenylation signal between those same sites. The resulting plasmid was named pTarget-3dPGKNeoVec-NX. This construct was linearized at the NotI site immediately upstream of the insulator sequence and dephosphorylated to prepare it for ligation to the 1.3 kb NotI fragment of pSA-IRESd2EGFP. The resulting plasmid was pHTP-GT.

The pHTPires2EGFP-GT gene trap vector was constructed from pHTP-GT. The ires2EGFP portion of pIRES2EGFP (Clontech) was excised by digestion with BamHI and XbaI. The approximately 1.3 kb fragment was ligated to BamHI/XbaI digested pHTP-GT, replacing the SA-IRESd2EGFP sequences between those same sites. The splice acceptor junction was recreated and modified to also contain an AscI site 5′ to the SA for insertion of additional sequence elements (e.g. recombinogenic elements, etc.) and an XbaI site 5′ to the BsgI site for use in sequence tag concatamerization as per the mage protocol. This was accomplished using synthetized sequences inserted as a double stranded DNA oligonucleotide containing EcoRI and BamHI adapter ends.

The pHTPires2EGFP-GT vector has been further modified to create pHTPfus1EGFP-GT, pHTPfus2EGFP-gt, and pHTPfus3EGFP by removal of the triple termination codons and IRES sequences and replacement with sequences encoding short runs of polyglycine in each of the three reading frames, respectively.

### EXAMPLE 2

This embodiment describes the establishment of gene trap cellular libraries using a gene-trap vector as described in Example 1. Gene trap cellular libraries were constructed in Jurkat cells, P19 EC cells or SF 268 glioma cells. The gene-trap vector was introduced by electroporation. Electroporation was performed using a BioRad Gene Pulser II se to 200 volts and 500 μF where 1×10⁷ cells were electorporated in a 1 ml volume containing between 40 and 60 μg of DNA. Cells were grown in the presence of G418 for a period of 10 days and surviving colonies were pooled. The number of colonies was approximately 1,500. Colonies were trypsinized using routine tissue culture methods and pooled to a tissue culture flask for additional culture. Cells were amplified by trypsinization and passage to additional culture flasks, retaining all of the resulting cells, until approximately 5×10⁷ cells were obtained. This population was then prepared for FACS by trypsinizing and filtering using standard protocols. When cells in which the gene-trap vector has been used to trap genes, are processed and subjected to FACS analysis, fluorescence distribution patterns (such as shown in FIG. 7) are generated. The fluorescent cells are then distributed into the wells of a matrix such that each well has one cell and each cell represents a unique trapped gene.

### EXAMPLE 3

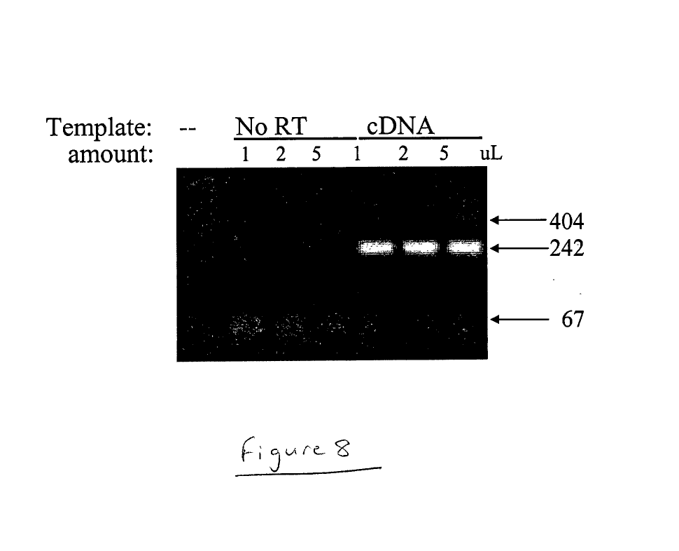

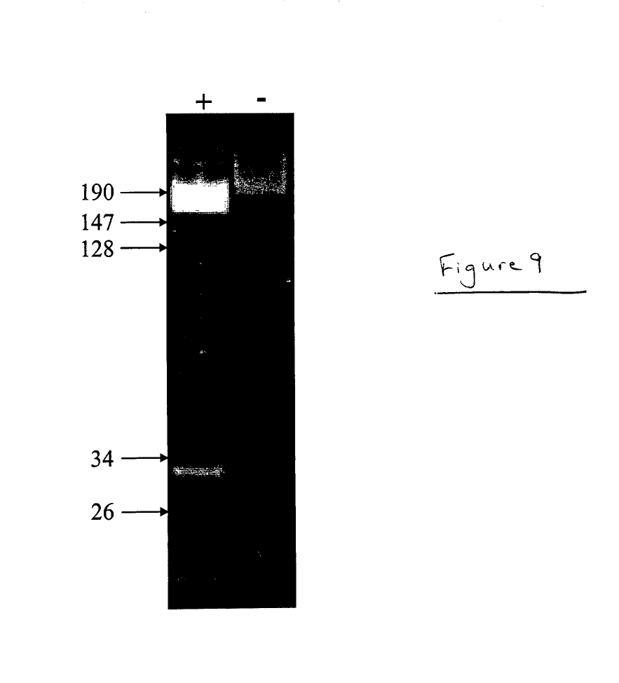

This embodiment demonstrates the generation of Assay Tag concatamers and describes the identification of Sequence Tags from trapped genes by MAGE from the splice donor junctions present in a small pool of P19 EC cell gene trap lines established as described in example 2. RNAs were isolated using GITC/phenol extraction and polyadenylated messages were selected on oligo dT cellulose by standard methods. First strand cDNA synthesis primed with oligo dT was performed using superscript II (Invitrogen) using standard conditions. A control sample in which reverse transcriptase was omitted was also prepared. RNA was hydrolyzed using NaOH, NaOH was neutralized and cDNAs were recovered by ethanol precipitation again using standard techniques. Second strand synthesis was primed using Biotinylated neotop2 primer (5′-B-CCGCTTTTCTGGATTCAT-3′ (SEQ ID NO:2)) and extended using the large fragment of E. coli DNA polymerase. Double stranded cDNA was digested with BpmI (New England BioLabs) as recommended by the manufacturer and incubated in streptavidin coated PCR tubes for 3 minutes at 37° C. Following binding tubes were washed 2 times with 150 μl of 150 mM NaCl in TE (10 mM Tris-HCl, pH 7.5, 1 mM EDTA). The MAGE universal adapter (5′-TCTAGAGGACTGCGTGGGCGA-3′ (SEQ ID NO:3); 5′-CCTCGCCCACGCAGTCCTCTAGANN-3′ (SEQ IS NO:4)) (16.6 nmoles) was added in 50 μl of ligation buffer plus 2 μl of T4 ligase and tubes were incubated for 2hours at 15° C. Following 2 washes as previous, 1 ul of 50 μM of each MAGE PCR primer (5′-CCTCGCCCACGCAGTCCTC-3′ (SEQ ID NO:5); 5′-CGGCTGGGTG TGGCGGAC-3′ (SEQ ID NO:9)) was added in 100 μl of Platinum Taq (Invitrogen) PCR reaction buffer containing 0.2 mM of each of dATP, dGTP, dCTP and dTTP, 2 mM MgCl₂ and 0.5 units of Platinum Taq polymerase. Thermal cycling was performed where 35 cycles of 94° C. for 0.75 minutes, 60° C. for 0.75 minutes and 72° C. for 0.75 minutes were used. Samples of the resulting PCR products were electrophoresed on an agarose gel as shown in FIG. 8. The predicted band of 232 bp is present in the lanes in which reverse transcriptase was present in the initial cDNA synthesis reaction but is absent from lanes where reverse transcriptase was omitted. The remaining PCR products were pooled, digested with XbaI and electorphoresed on an 8% polyacrylamide as shown in FIG. 9. The predicted 32 nt fragment containing the sequence tags was isolated and incubated in 20 μl of T4 DNA ligation buffer for between 20 and 60 minutes prior to addition of Xba cut and de-phosphorylated BlueScript vector. Ligations were continued for an additional 60 minutes and used for transformation of competent HB101. Transformed colonies of HB101 were selected and used in preparation of DNA for sequencing by standard techniques.

Sequencing revealed that the concatamers ranged from 2 to 8 repeats in this experiment and consisted of the predicted vector/universal primer sequences separated by 16 nucleotide long tags. Blast searches of the tags revealed four unknown sequences (i.e. not present in the NCBI mouse EST or non-redundant sequence databases) and four known sequences comprising predicted exons from albumin (TTTCTCAGGGTAGCCT; SEQ ID NO:10), HSP84 (AGCTTTGAATTCATGA; SEQ ID NO:11), actin binding protein (ACTACATCTCCTCCCT; SEQ ID NO:12) and erythroid differentiation regulatory protein (GGCGACACGCGCACCT; SEQ ID NO:13).

### EXAMPLE 4

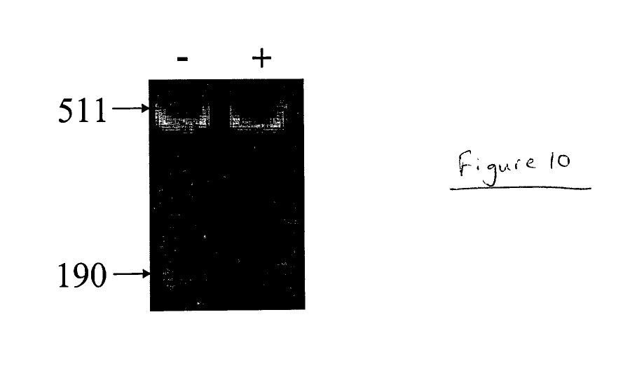

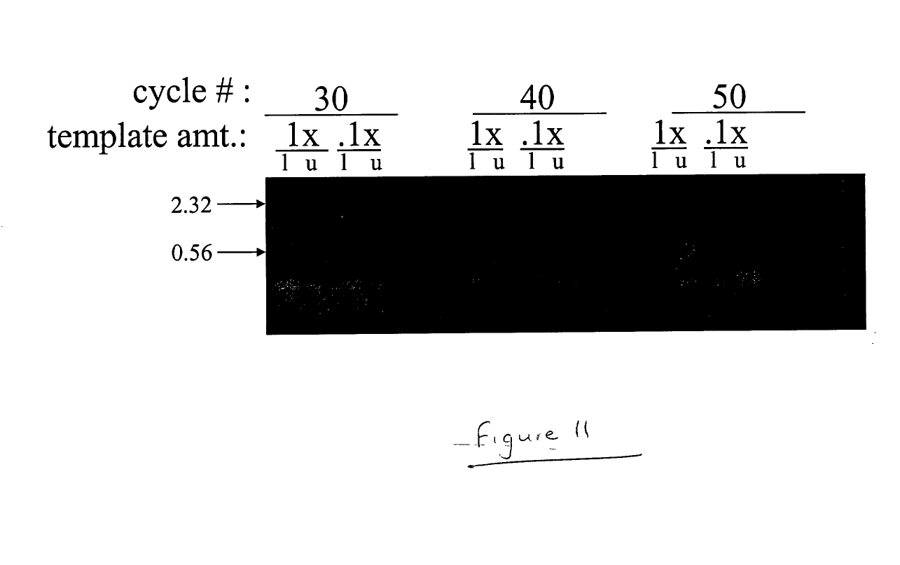

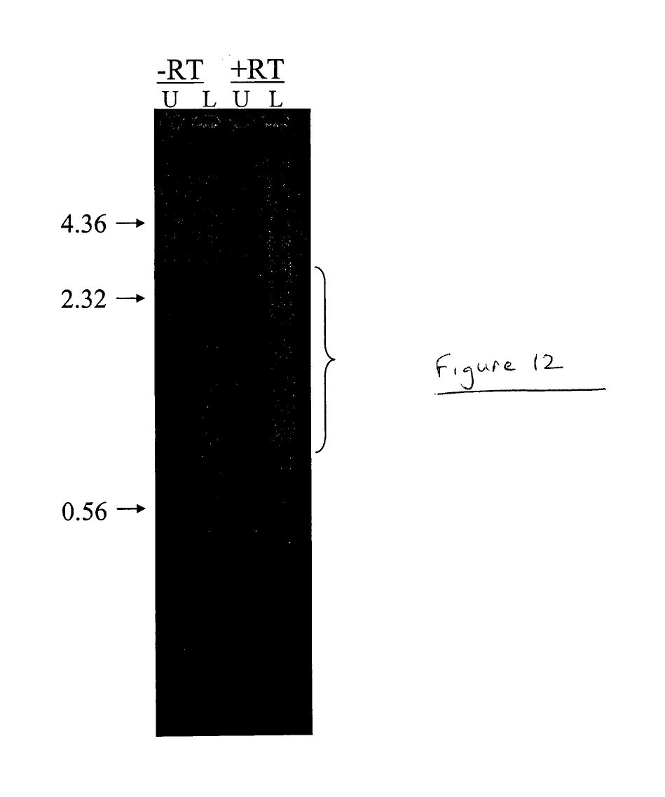

This embodiment illustrates the principle of self-amplifying MAGE (SA-MAGE) on a known template DNA. To demonstrate the ability of the PCR to generate concatamers from a directly repeated sequence separated by a non-repeated sequence as illustrated in FIG. 3, fragments of the gene trap vector shown in FIG. 1A were generated by BmpI digestion and samples which were either ligated or not ligated to the SA-MAGE adapter (5′-TTCTCTAGACAGTCTGGAG-3′(SEQ ID NO: 6); 5′-CTCCAGACTGTCTAGAGAANN-3′ (SEQ ID NO:7)) were re-digested with SstI and a fragment from the BmpI site, adjacent to the splice donor, to an SstI site was isolated for both the ligated and non-ligated material. The presence of the adapter on the ligated fragment was confirmed as shown in FIG. 10. These fragments were used as templates at two different concentration (1× or 0.1×) in PCR reactions with the SA-MAGE primers (5′-TTCTCTAGACAGTCTGGAC-3′ (SEQ ID NO:6); 5′-CTCCAGACTGTCTAGAGAA-3′ (SEQ ID NO:8)) shown in FIG. 11. PCR was performed using either 30, 40, or 50 cycles of 94° C. for 0.5 minutes, 45° C. for 0.5 minutes and 72° C. for 1.0 minutes. Products resulting from the PCR reactions were electrophoresed on a 1.5% agarose gel as shown in FIG. 12. The presence of high molecular weight DNA in the ligated (l) but not un-ligated (u) template lanes demonstrates that the presence of a direct repeat in the template DNA, that also repeated in the top and bottom strand primers, results in formation of a concatamerized product during PCR. The template concentration dependence of the concatamerization reaction is also consistent with a requirement to utilize the majority of the primers in generation of monomer length repeats before the concatamerization reaction occurs efficiently. These observations demonstrate the principle on which SA-MAGE is based.

### EXAMPLE 5

This embodiment demonstrates the generation of Assay Tag concatamers and describes the identification of Sequence Tags by SA-MAGE from the splice donor junctions present in a small pool of P19 EC cell gene trap lines established as described in example 2. The cDNA used in this demonstration was identical to that used in Example 3 through the point at which streptavidin coated PCR tubes containing BpmI digested cDNAs were washed. Subsequent to this step the SA-MAGE adapter (SEQ ID NO:6,7) shown in was substituted for the MAGE adapter (SEQ ID NO:3,4) in the ligation reaction as described in Example 3. Control samples in which reverse transcriptase was omitted in the first round cDNA synthesis, or ligase was omitted in the SA-MAGE adapter ligation step, or both were also included. Following ligation two rounds of PCR as described in Example 4 were performed using the SA-MAGE primers (SEQ ID NO: 6,8). Products from each of the PCR reactions were electrophoresed on a 1.5% agarose gel as shown in FIG. 12. The presence of high molecular weight DNA in the lane in which both reverse transcriptase and ligase were included, but absence of this product in the control lanes, demonstrates the formation of concatamers during the PCR step. Another embodiment of this method would be the inclusion of a low concentration of primers carrying a restriction endonuclease cleavage site to facilitate cloning the concatamers.

Various examples have been described herein for the purpose of illustration. Modifications within the purview of those skilled in the art can be made without departing from the scope of the invention as described herein.

What is claimed is:

1 A method for identification of Sequence Tags from trapped genes, comprising the steps of:
a) providing a gene-trap vector comprising a splice donor, a type IIS restriction endonuclease cleavage site and either a splice donor or a polyadenylation site;
b) preparing mRNA from cells stably transfected with the gene-trap vector;
c) synthesizing a first and second cDNA strands from the mRNA;
d) digesting with restriction endonucleases including the type IIS restriction endonucleases to produce Assay Tags, wherein each Assay Tag comprises a Sequence Tag and a portion of the gene-trap vector;
f) concatenating the Assay Tags;
g) amplifying and sequencing the concatamers to identify the sequence of the Assay Tags and the Sequence Tags.

2. The method of claim 1, wherein the second cDNA strand is biotinylated.

3. The method of claim 1, wherein the type IIS restriction endonuclease is selected from the group consisting of BsgI, BpmI, BsmF1, MmeI and Fok1.

4. A method for rapid analysis of gene expression comprising the steps of:
a) providing a gene-trap vector comprising a splice acceptor, a sequence encoding a fluorescent reporter protein, a type IIS restriction endonuclease cleavage site, and a splice donor or a polyadenylation site wherein the fluorescent reporter protein directly or indirectly produces fluorescence;
b) stably transfecting a population of cells with the gene-trap vector encoding a fluorescent reporter protein;
c) sorting the population of stably transfected cells by FACS to identify cells that express the fluorescent reporter protein, wherein expression of the reporter protein is indicative of the expression of the trapped gene;
d) distributing and expanding the stably transfected cells expressing the trapped gene in wells provided in a matrix format, wherein each well represents one trapped gene;
e) pooling cells from discrete wells in the matrix to represent pooling in x y and z axis;
f) preparing mRNA from the pooled cells;
g) synthesizing a first cDNA strand from the mRNA followed by synthesis of a second cDNA strand, wherein the second cDNA strand is linked to an anchor molecule;
h) digesting with the type IIS restriction endonucleases to produce Assay Tags comprising a Sequence Tag from the trapped gene and a portion of the gene-trap vector;
i) concatenating the Assay Tags from each pool;
j) cloning and sequencing the concatamers from each pool;
k) identifying the location of a sequence tag within the matrix by its unique presence in the discrete pools.

5. The method of claim 4, wherein the reporter protein is the green fluorescent protein.

6. The method of claim 4, wherein the reporter protein is the enhanced green fluorescent protein.

7 The method of claim 4, wherein the recognition endonucleases are BmpI and BsgI.

8. The method of claim 4, wherein the second strand of cDNA is linked to an anchor molecule;

9. The method of claim 4, wherein the anchor molecule is biotin.

10. A method for rapid analysis of gene expression comprising the steps of:
a) providing a gene-trap vector comprising a splice acceptor, a sequence encoding a fluorescent reporter protein, a type IIS restriction endonuclease cleavage site, and a splice donor or a polyadenylation site wherein the fluorescent reporter protein directly or indirectly produces fluorescence;
b) stably transfecting a population of cells with the gene-trap vector encoding a fluorescent reporter protein;
c) sorting the population of stably transfected cells by FACS to identify cells that express the fluorescent reporter protein, wherein expression of the reporter protein is indicative of the expression of the trapped gene;
d) expanding the stably transfected cells;
e) preparing mRNA from the expanded cells;
f) synthesizing a first cDNA strand from the mRNA followed by synthesis of a second cDNA strand, wherein the second cDNA strand is linked to an anchor molecule;
g) digesting with restriction endonucleases, wherein one of the endonucleases is the type IIS restriction endonucleases to produce Assay Tags comprising a Sequence Tag from the trapped gene and a portion of the gene-trap vector;
h) concatenating the Assay Tags from each pool;
i) cloning and sequencing the concatamers to obtain the sequence of the sequence tags.

11. The method of claim 10, wherein the reporter protein is the green fluorescent protein.

12. The method of claim 10, wherein the reporter protein is the enhanced green fluorescent protein.

13 The method of claim 10, wherein the recognition endonucleases are BmpI and BsgI.

14. The method of claim 10, wherein the second strand of cDNA is linked to an anchor molecule;

15. The method of claim 10, wherein the anchor molecule is biotin.

16. A nucleic acid construct comprising in downstream sequence: a splice acceptor, a type II restriction endonuclease cleavage site; and a splice donor or a polyadelylation site to terminate transcription.

17. The nucleic acid construct of claim 16 comprising in downstream sequence: a splice acceptor, a first type IIS restriction endonuclease cleavage site, termination codons in all three reading frames, an internal ribosome entry site, a promotorless coding sequence encoding a polypeptide providing a positive or negative selection traits, a polyadynylation signal to terminate transcription, a promoter, a selectable marker, a second type IIS restriction endonuclease cleavage site, and a splice donor.

18. The nucleic acid construct of claim 17 comprising in downstream sequence: a recombinogenic sequence, a splice acceptor, a first type IIS restriction endonuclease cleavage site, termination codons in all three reading frames, an internal ribosome entry site, a promotorless coding sequence encoding a polypeptide providing a positive or negative selection traits, a polyadynylation signal to terminate transcription, an insulator sequence, a promoter, a recombinogenic sequence, a selectable marker, a second type IIS restriction endonuclease cleavage site, and a splice donor.

19. The nucleic acid construct of claim 17, wherein the first IIS restriction endonuclease cleavage site is BsgI and the second IIS restriction endonuclease cleavage site is BpmI.

20. The nucleic acid construct of claim 118, wherein the polypeptide providing a positive or negative selection trait is a fluorescent protein.

21. The nucleic acid construct of claim 20, wherein the fluorescent protein is enhanced green fluorescence protein.

22. A kit for rapid analysis of gene expression comprising: a gene-trap vector of claim 16, a type IIS endonucleases and one or more primers of SEQ ID NO: 2-9.
